(19) United States
(12) Patent Application Publication
(10) Pub. No.: US 2026/0315035 A1
(43) Pub. Date: Oct. 15, 2026

(54) AUTOMATICALLY SELECTING VIEWS AND CAPTURING ULTRASOUND IMAGES IN POINT OF CARE ULTRASOUND

(71) Applicant: EchoNous, Inc., Redmond, WA (US)

(72) Inventors: Babajide Ayinde, Redmond, WA (US); Matthew Cook, Redmond, WA (US)

(21) Appl. No.: 19/179,564

(22) Filed: Apr. 15, 2025

(51) Int. Cl. A61B 8/00; G06N 20/00; G16H 30/20

(52) U.S. Cl. CPC A61B 8/5215; G06N 20/00; G16H 30/20

(57) ABSTRACT

A facility for automatically selecting views and capturing ultrasound images is described. The facility captures ultrasound echo data comprising a sequence of ultrasound images and applies one or more machine learning models to one or more first images of the sequence of ultrasound images. The facility identifies a first view of a diagnostic protocol based on the output of the one or more machine learning models. The facility generates one or more directions to capture an ultrasound images associated with the first view based on the output of the one or more machine learning models. The facility displays an indication of the generated one or more directions to capture the ultrasound image associated with the first view.

### BACKGROUND

Ultrasound imaging is a useful medical imaging modality. For example, internal structures of a patient's body may be imaged before, during or after a therapeutic intervention. Also, multiple views may be obtained as part of ultrasound imaging to aid in diagnosing a patient, such as when the ultrasound imaging is to follow one or more diagnostic protocols.

A healthcare professional typically holds a portable ultrasound probe, sometimes called a “transducer,” in proximity to the patient and moves the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired presentation on a screen, such as a two-dimensional cross-section of a three-dimensional volume.

Particular views of an organ or other tissue or body feature (such as fluids, bones, joints or the like) can be clinically significant. Such views may be prescribed by clinical standards as views that should be captured by the ultrasound operator, depending on the target organ, diagnostic purpose, or the like.

Clinical diagnostic protocols (sometimes referred to as “diagnostic studies” or “studies”) specify a way of collecting and analyzing information about a patient to determine a likely diagnosis of a medical issue. Some such protocols specify ways of collecting ultrasound images and/or videos from particular views; identifying certain visual features in this ultrasound data; and measuring or otherwise assessing those visual features. Furthermore, some such protocols specify multiple views for which ultrasound data is to be obtained.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration of a physiological sensing device, in accordance with one or more embodiments of the present disclosure.

FIG. 2 is a block diagram showing some of the components typically incorporated in at least some of the computer systems and other devices on which the facility operates.

FIG. 3 is a flow diagram showing a sample process to use one or more machine learning models to guide a clinician to an anatomical window and automatically capture one or more views, performed by the facility in some embodiments.

FIG. 4 is a model architecture diagram showing the organization of a classification machine learning model for detecting an anatomical window (an “anatomical window machine learning model”), used by the facility in some embodiments.

FIG. 5 is a flow diagram showing a sample process to use an anatomical window machine learning model to direct a clinician to an anatomical window, used by the facility in some embodiments.

FIG. 6 is a model architecture diagram showing the organization of a classification machine learning model for guiding a clinician to produce an ultrasound image (a “guidance machine learning model”), used by the facility in some embodiments.

FIG. 7 is a flow diagram showing a sample process to use a guidance machine learning model to guide a clinician to produce an ultrasound image, used by the facility in some embodiments.

FIG. 8A is a sample ultrasound image for training a guidance machine learning model, used by the facility in some embodiments.

FIG. 8B is a sample ultrasound image for training a guidance machine learning model, used by the facility in some embodiments.

FIG. 9 is a model architecture diagram showing the organization of a classification machine learning model for grading an ultrasound image (a “grading machine learning model”), used by the facility in some embodiments.

FIG. 10 is a flow diagram showing a sample process to use a grading machine learning model for automatically capturing an ultrasound image, used by the facility in some embodiments.

FIG. 11 is a model architecture diagram showing the organization of a combination grading and guidance classification machine learning model, used by the facility in some embodiments.

FIG. 12 is a sample ultrasound image output by the facility for directing a clinician in some embodiments.

### DETAILED DESCRIPTION

Some ultrasound diagnostic protocols are cardiac diagnostic protocols, which are designed to assess a patient's heart, and aid in the evaluation of the patient's cardiac system. Cardiac diagnostic protocols may include obtaining views representing the Left Ventricle, end-diastolic volume, end-systolic volume, Apical 4 Chamber (A4C), Ejection Fraction, other aspects of a patient's cardiovascular system, or some combination thereof. The cardiac diagnostic protocols may require one or more views of a patient's cardiac system such as: A4C (Apical 4-Chamber), A2C (Apical 2-Chamber), A5C (Apical 5-Chamber), A3C (Apical 3-Chamber), PLAX (Parasternal Long Axis), SUBCOSTAL-4C (Subcostal 4-Chamber), PSAX-AV (Parasternal Short-Axis View at the Aortic Valve level), PSAX-AP (Parasternal Short-Axis View at the Apex level), PSAX-MV (Parasternal Short-Axis View at the Mitral Valve level), PSAX-PM (Parasternal Short-Axis View at the Papillary Muscle level), SUBCOSTAL-IVC (Subcostal Inferior Vena Cava View), RVIT (Right Ventricular Inflow Tract View), RVOT (Right Ventricular Outflow Tract View). For example a cardiac diagnostic protocol for calculating a patient's Left Ventricular Ejection Fraction by using the Simpson's Biplane Method may involve the acquisition of the A2C view and the A4C view. As another example, a standard echocardiogram may involve the acquisition of each of the views listed above.

The inventors have recognized that it is difficult and time-consuming for clinicians to manipulate aspects of an ultrasound device, such as an ultrasound transducer, to obtain views for particular ultrasound diagnostic protocols that can be used to diagnose a patient. It is difficult for clinicians to ensure that ultrasound echo data received by the ultrasound device represents an area that is spatially near a view that the clinician is trying to obtain for a particular ultrasound diagnostic protocol. Additionally, it is difficult for clinicians to ensure that a view is properly captured, or that their movement of aspects of an ultrasound device will result in a properly captured view, such that the view can be used to aid in the patient's diagnosis.

Furthermore, while some conventional ultrasound systems are able to indicate a region of the patient's body or direct a clinician in the motion of an ultrasound transducer, such conventional ultrasound systems must be re-configured by the clinician after each view in order to obtain the next view for the diagnostic protocol. Conventional ultrasound systems are also unable to determine whether a view captured by a clinician is a view that can be used to aid in a patient's diagnosis. Thus, in cases where a diagnostic protocol requires multiple views, conventional systems are not able to automatically select 1) a next view for the clinician and 2) re-configure the ultrasound system such that it can direct a clinician to the next view and capture the view. Additionally, conventional ultrasound systems are unable to allow a clinician to freely operate an ultrasound device such that the clinician is able to be guided to any of the views associated with a diagnostic protocol.

In response to recognizing these disadvantages, the inventors have conceived and reduced to practice a software and/or hardware facility for selecting views and automatically capturing ultrasound images associated with the views (“the facility”). In particular, the facility applies one or more machine learning models to ultrasound images sensed by an ultrasound system and captures an ultrasound image determined by the one or more machine learning models to constitute an adequate version of one of the views included in the diagnostic protocol. Based on capturing the ultrasound image, the facility selects a second view associated with the diagnostic protocol for which no ultrasound image has been captured.

In some embodiments, the one or more machine learning models used by the facility include: a machine learning model configured to output a score representing the degree to which an ultrasound image indicates an optimal view for a diagnostic protocol (a “grading machine learning model”); a machine learning model configured to output whether a manipulation of a sensing device likely to produce a more optimal version of a view to which an ultrasound image corresponds (a “guidance machine learning model”); a grading and guidance machine learning model that combines both of the foregoing models in a single model; a machine learning model configured to output whether an ultrasound image comprising an image was captured near an anatomical window (an “anatomical window machine learning model”); or some combination thereof. An “optimal view” may be a view that is able to be used to properly diagnose a patient. A “sub-optimal view” may be a view that may not always be able to be used to properly diagnose a patient, and which is within a threshold range of being an optimal view. For example, a sub-optimal view may be a view in which a change in the position of an ultrasound transducer through one or more movements, such as rocking, sliding, tilting, rotation, etc., may produce an optimal view. In some embodiments, the one or more machine learning models are machine learning models that are trained based on one or more diagnostic protocols, one or more views, one or more anatomical windows, or some combination thereof. Thus, in such embodiments, a first set of machine learning models may be configured to produce output related to a first diagnostic protocol, a second set of machine learning models may be configured to produce output related to a second diagnostic protocol, etc.

In some embodiments, the output of an anatomical window machine learning model includes directions to manipulate an aspect of an ultrasound device, such as an ultrasound transducer, towards, or away from, an anatomical window associated with a view for a diagnostic protocol. An anatomical window is a region of a patient's body within which one or more views associated with a diagnostic protocol are to be obtained. Thus, in some embodiments, the output of the anatomical window machine learning model may be used to determine whether an ultrasound image was captured spatially near a view associated with a diagnostic protocol.

In some embodiments, the output of a guidance machine learning model includes directions regarding how to manipulate an aspect of an ultrasound device to obtain an optimal version of a view associated with a diagnostic protocol. Manipulations of an aspect of an ultrasound device, such as an ultrasound transducer, may include changes in direction of movement, distance of movement, rotation or other orientation of a probe, depth settings, or other manipulations of an aspect of an ultrasound transducer.

The facility receives a selection of a diagnostic protocol, such as via user input, and selects a set of machine learning models based on the diagnostic protocol. In some embodiments, the set of machine learning models includes one or more anatomical window machine learning models, one or more guidance machine learning models, one or more grading machine learning models, one or more guidance and grading machine learning models, or some combination thereof. In some embodiments, the facility applies at least one of the selected machine learning models to a sensed ultrasound image to determine whether the ultrasound image was sensed near a location of a view that is to be obtained for the diagnostic protocol. In some embodiments, based on a determination that the ultrasound image was sensed near the view, the facility applies at least one of the selected machine learning models to guide the clinician to obtain one or more ultrasound images for the view. In some embodiments, the facility applies at least one of the selected machine learning models to one or more ultrasound images obtained for the view to determine whether to “capture” the view. In some embodiments, based on a determination that the view is to be captured, the facility automatically stores the ultrasound images and associates them with the view.

The facility selects a second view associated with the diagnostic protocol based on capturing a first view of the diagnostic protocol. In some embodiments, the facility selects the second view based on one or more remaining views associated with the diagnostic protocol that have yet been captured. In some embodiments, as part of selecting the second view, the facility selects a second set of machine learning models associated with the second view. In some embodiments, the second set of machine learning models include one or more machine learning models selected for capturing the first view of the diagnostic protocol.

Although the views and diagnostic protocols described in this specification are directed to a patient's cardiac system, embodiments are not so limited, and the facility may be configured to obtain views for diagnostic protocols related to other parts of a patient's body, such as abdominal diagnostic protocols (for example, the liver, kidney, spleen, etc.), lung diagnostic protocols (for example, pleural effusion, pneumothorax assessment, etc.), obstetric views (for example, fetal measurements, placental assessment, etc.), other parts or systems of a patients body for which ultrasound systems may be used to obtain views or used as part of a diagnostic protocol, or some combination thereof.

By performing in some or all of the ways described above, the facility is able to automatically select and capture views associated with one or more diagnostic protocols, without requiring the clinician to re-configure the ultrasound device. Furthermore, the facility is able to allow the clinician to freely operate the ultrasound device and direct the clinician to views associated with one or more diagnostic protocols selected by the clinician.

Also, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by guiding the clinician to views, the facility reduces the amount of time the ultrasound system is occupied, and ultrasound images sensed relative to the clinician's freely operating the ultrasound device. As another example, by automatically selecting and capturing views and guiding the clinician in the capture of views, the facility is able to reduce the number of captured views that are not able to be used to diagnose the patient, thus reducing the amount of memory needed to store captured views, total utilization time of this device, etc.

Further, for at least some of the domains and scenarios discussed herein, the processes described herein as being performed automatically by a computing system cannot practically be performed in the human mind, for reasons that include that the starting data, intermediate state(s), and ending data are too voluminous and/or poorly organized for human access and processing, and/or are a form not perceivable and/or expressible by the human mind; the involved data manipulation operations and/or subprocesses are too complex, and/or too different from typical human mental operations; required response times are too short to be satisfied by human performance; etc. For example, the ultrasound echo data generated by an ultrasound system is in a format which a human is unable to comprehend without first using specialized computing systems to generate an ultrasound image from the ultrasound echo data.

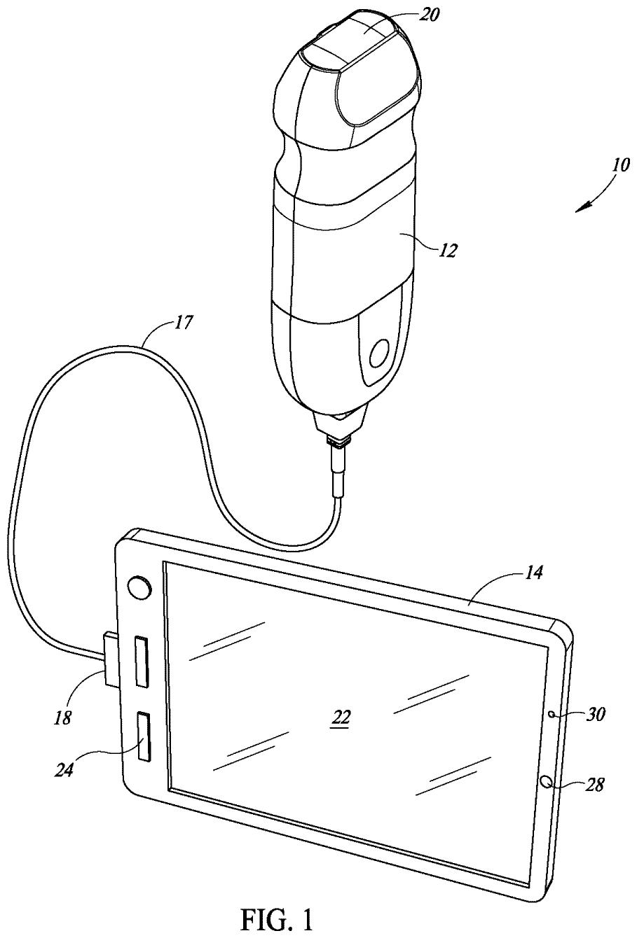

FIG. 1 is a schematic illustration of a physiological sensing device 10, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from a user that touches the screen. In such embodiments, the user interface 24 may include a portion or the entire display screen 22, which is capable of receiving user input via touch. In some embodiments, the user interface 24 may include one or more buttons, knobs, switches, and the like, capable of receiving input from a user of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

The probe 12 includes a housing, which forms an external portion of the probe 12. The housing includes a sensor portion located near a distal end of the housing, and a handle portion located between a proximal end and the distal end of the housing. The handle portion is proximally located with respect to the sensor portion.

The handle portion is a portion of the housing that is gripped by a user to hold, control, and manipulate the probe 12 during use. The handle portion may include gripping features, such as one or more detents, and in some embodiments, the handle portion may have a same general shape as portions of the housing that are distal to, or proximal to, the handle portion.

The housing surrounds internal electronic components and/or circuitry of the probe 12, including, for example, electronics such as driving circuitry, processing circuitry, oscillators, beamforming circuitry, filtering circuitry, and the like. The housing may be formed to surround or at least partially surround externally located portions of the probe 12, such as a sensing surface. The housing may be a sealed housing, such that moisture, liquid or other fluids are prevented from entering the housing. The housing may be formed of any suitable materials, and in some embodiments, the housing is formed of a plastic material. The housing may be formed of a single piece (e.g., a single material that is molded surrounding the internal components) or may be formed of two or more pieces (e.g., upper and lower halves) which are bonded or otherwise attached to one another.

In some embodiments, the probe 12 includes a motion sensor. The motion sensor is operable to sense a motion of the probe 12. The motion sensor is included in or on the probe 12 and may include, for example, one or more accelerometers, magnetometers, or gyroscopes for sensing motion of the probe 12. For example, the motion sensor may be or include any of a piezoelectric, piezoresistive, or capacitive accelerometer capable of sensing motion of the probe 12. In some embodiments, the motion sensor is a tri-axial motion sensor capable of sensing motion about any of three axes. In some embodiments, more than one motion sensor 16 is included in or on the probe 12. In some embodiments, the motion sensor includes at least one accelerometer and at least one gyroscope.

The motion sensor may be housed at least partially within the housing of the probe 12. In some embodiments, the motion sensor is positioned at or near the sensing surface of the probe 12. In some embodiments, the sensing surface is a surface which is operably brought into contact with a patient during an examination, such as for ultrasound imaging or auscultation sensing. The ultrasound sensor 20 and one or more auscultation sensors are positioned on, at, or near the sensing surface.

In some embodiments, the transducer array of the ultrasound sensor 20 is a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens, which may be positioned over the transducer array, and which may form a part of the sensing surface. The focusing lens may be any lens operable to focus a transmitted ultrasound beam from the transducer array toward a patient and/or to focus a reflected ultrasound beam from the patient to the transducer array. The ultrasound focusing lens may have a curved surface shape in some embodiments. The ultrasound focusing lens may have different shapes, depending on a desired application, e.g., a desired operating frequency, or the like. The ultrasound focusing lens may be formed of any suitable material, and in some embodiments, the ultrasound focusing lens is formed of a room-temperature-vulcanizing (RTV) rubber material.

In some embodiments, first and second membranes are positioned adjacent to opposite sides of the ultrasound sensor 20 and form a part of the sensing surface. The membranes may be formed of any suitable material, and in some embodiments, the membranes are formed of a room-temperature-vulcanizing (RTV) rubber material. In some embodiments, the membranes are formed of a same material as the ultrasound focusing lens.

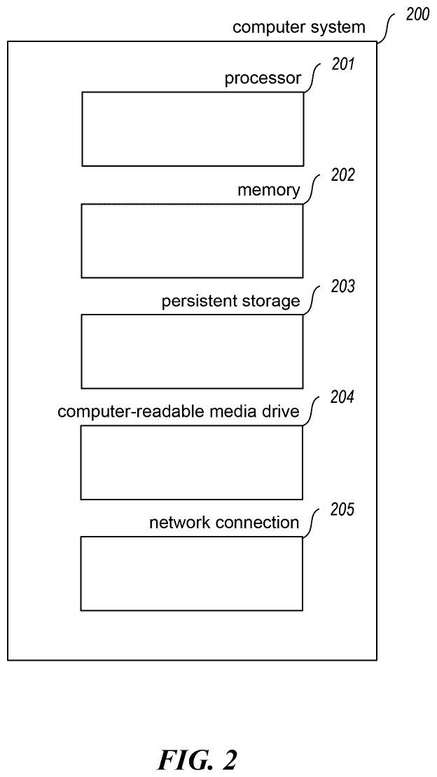

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components. None of the components shown in FIG. 2 and discussed above constitutes a data signal per se.

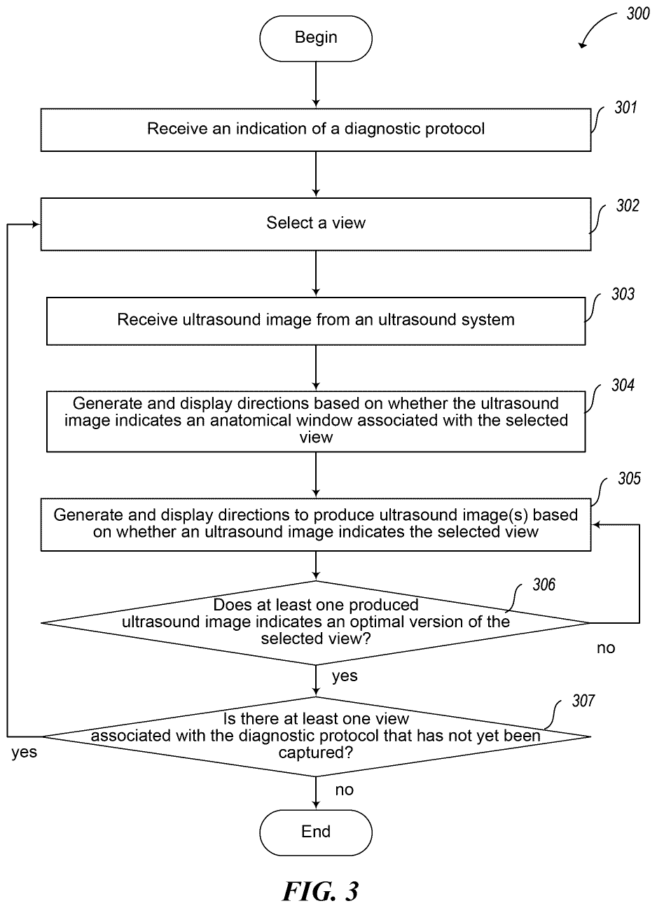

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc. In various embodiments, the facility performs the acts shown in FIG. 3 and in each of the flow diagrams discussed below, with respect to ultrasound echo data of various types, including images, videos, sequences of images, other ultrasound artifacts or ultrasound echo data, or some combination thereof.

FIG. 3 is a flow diagram showing a sample process 300 using one or more machine learning models to guide a clinician to an anatomical window and automatically capture one or more views, performed by the facility in some embodiments.

First, at act 301, the facility receives an indication of a diagnostic protocol. In some embodiments, the facility receives the indication of a diagnostic protocol via user input. In some embodiments, the facility selects the diagnostic protocol based on data indicating one or more studies, diagnoses, tests, other medical data, or some combination thereof, associated with a patient.

At act 302, the facility selects a view associated with the diagnostic protocol. In some embodiments, the facility selects a view based on a determination that an ultrasound image has not been captured for one or more views associated with the diagnostic protocol. In some embodiments, the facility selects a view based on a determination that an ultrasound image that indicates a part of a body that is spatially nearer to a part of a body associated with a first view than to a part of a body associated with another view. In some embodiments, the facility selects a view based on a pre-determined order for capturing views for the diagnostic protocol.

At act 303, the facility receives an ultrasound image from an ultrasound system. In some embodiments, the facility receives multiple ultrasound images from an ultrasound system at various times throughout the performance of the process 300. In such embodiments, the facility may perform one or more acts of the process 300 with respect to at least the most recently received ultrasound image.

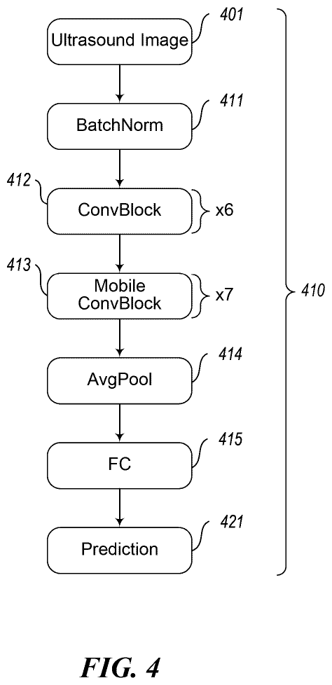

At act 304, the facility generates and displays directions to an operator of the ultrasound system based on whether the ultrasound image indicates an anatomical window associated with the selected view. In some embodiments, the facility performs act 304 by applying an anatomical window machine learning model, such as the anatomical window machine learning model 410 described below with respect to FIG. 4, to the received ultrasound image.

Those skilled in the art will appreciate that a variety of neural network types and particular architectures may be straightforwardly substituted for the architecture shown in FIG. 4, and in the additional architecture diagrams discussed below.

FIG. 4 is a model architecture diagram showing the organization of a classification machine learning model for detecting an anatomical window 410 (an “anatomical window machine learning model”), used by the facility in some embodiments. The anatomical window machine learning model 410 is made up of a batch normalization block 411, one or more convolutional blocks 412, one or more mobile convolutional blocks 413, an average pool block 414, and a fully connected block 415. In some embodiments, the anatomical window machine learning model 410 includes a convolutional neural network feature extractor (not shown) that extracts one or more features of ultrasound images received by the anatomical window machine learning model 410 as input.

The anatomical window machine learning model 410 may receive an ultrasound image 401 as input. In some embodiments, the anatomical window machine learning model 410 receives one or more other types of ultrasound echo data as input, such as images, videos, sequences of images, other ultrasound artifacts or ultrasound echo data, or some combination thereof.

The anatomical window machine learning model 410 outputs a prediction 421. In some embodiments, the prediction 421 may include a prediction of an anatomical window depicted by the ultrasound image, a prediction of one or more views associated with a diagnostic protocol that are spatially near or within the anatomical window, or some combination thereof. For example, the anatomical window machine learning model 410 may indicate that the ultrasound system that provided the input ultrasound image 401 is scanning a patient's heart, abdomen, lungs, or other regions of the patient's body. In such an example, the output of the anatomical window machine learning model 410 may also include a prediction of a view associated with a diagnostic protocol that is spatially nearest to the current location within the patient's body that is being scanned by the ultrasound system.

In some embodiments, the anatomical window machine learning model 410 is trained by using a set of ultrasound images that depict anatomical windows, one or more anatomical windows depicted by each ultrasound image of the set of ultrasound images, one or more views that are spatially near or within an anatomical window depicted by each ultrasound image of the set of ultrasound images, or some combination thereof.

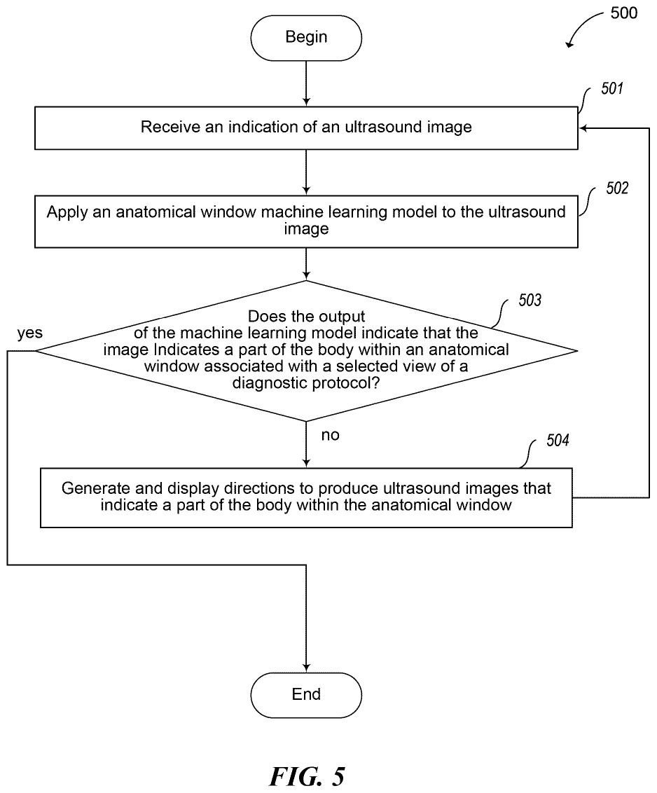

In some embodiments, the facility may apply an anatomical window machine learning model to the ultrasound image by using the process 500, described below in connection with FIG. 5.

FIG. 5 is a flow diagram showing a sample process 500 using an anatomical window machine learning model to direct a clinician to an anatomical window, used by the facility in some embodiments.

First, at act 501, the facility receives an indication of an ultrasound image.

At act 502, the facility applies an anatomical window machine learning model to the ultrasound image.

At act 503, the facility determines whether the output of the anatomical window machine learning model indicates that the ultrasound image indicates a part of the body within an anatomical window associated with a selected view of a diagnostic protocol. If the ultrasound image depicts an anatomical window associated with the selected view, the process 500 ends, otherwise, the process 500 proceeds to act 504.

At act 504, the facility generates and displays directions to produce ultrasound images that indicate a part of the body within the anatomical window. In some embodiments, the facility generates the directions based on the output of the anatomical window machine learning model. After act 504, the process 500 proceeds to act 501.

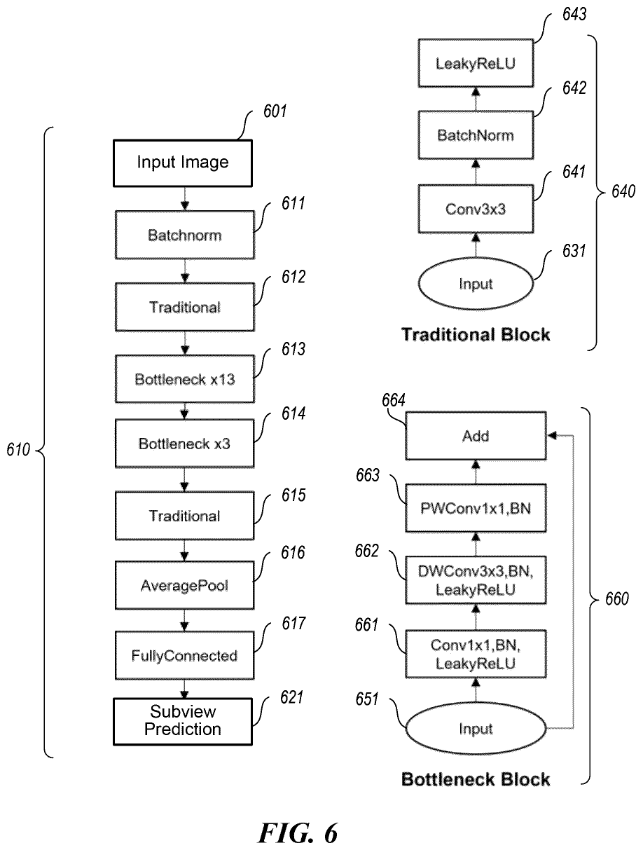

Returning to FIG. 3, at act 305, the facility generates and displays directions to produce at least one ultrasound image based on whether an ultrasound image indicates the selected view. In some embodiments, the perform act 305, the facility applies a guidance machine learning model, such as the guidance machine learning model 610 described below in connection with FIG. 6, to the ultrasound image.

FIG. 6 is a model architecture diagram showing the organization of a classification machine learning model for guiding a clinician to produce an ultrasound image 610 (a “guidance machine learning model”), used by the facility in some embodiments. The guidance machine learning model 610 includes a batch normalization block 611, one or more traditional blocks 612 and 615, one or more bottleneck blocks 613 and 614, an average pooling layer 616, and a fully connected layer 617. The traditional blocks 612 and 615 may each comprise a traditional block 640. The traditional block 640 includes an input 631, a convolutional block 641, a batch normalization block 642, and a leaky ReLU activation function block 643. The bottleneck blocks 613 and 614 may each comprise a bottleneck block 660. The bottleneck block 660 includes an input 651, a convolutional bottleneck and leaky ReLU activation function layer 661, a depthwise convolutional bottleneck and leaky ReLU activation function layer 662, a pointwise convolutional bottleneck layer 663, and one or more additional layers 664. In some embodiments, the guidance machine learning model 610 includes a convolutional neural network feature extractor (not shown) that extracts one or more features of ultrasound images received by the guidance machine learning model 610 as input.

The guidance machine learning model 610 may receive an ultrasound image 601 as input. In some embodiments, the guidance machine learning model 610 receives one or more other types of ultrasound echo data as input, such as images, videos, sequences of images, other ultrasound artifacts or ultrasound echo data, or some combination thereof.

The guidance machine learning model 610 outputs a prediction 621. In some embodiments, the prediction 621 may include a prediction of a view depicted by an ultrasound image, a prediction of whether a more optimal version of the view can be obtained, a prediction of one or more manipulations of an ultrasound sensing device for obtaining a more optimal version of the view, or some combination thereof. In some embodiments, a manipulation of an ultrasound sensing device may include changes in direction of movement, distance of movement, rotation or other orientation of a probe, depth settings, or other manipulations of an aspect of an ultrasound system or transducer.

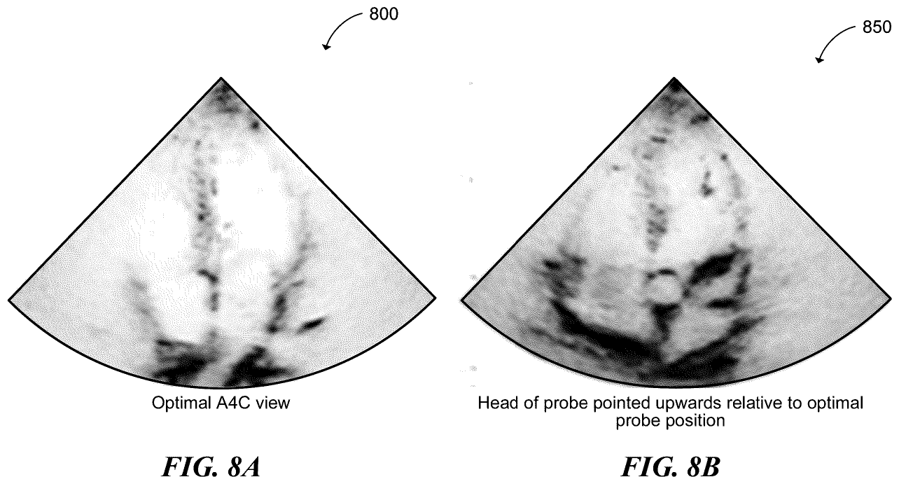

In some embodiments, the guidance machine learning model 610 is trained by using one or more ultrasound images that indicate an optimal version of a view, one or more ultrasound images that indicate a sub-optimal version of the view, one or more descriptions of a manipulation of a transducer from a position or settings that would yield an optimal version of the view to a position or settings that would yield a sub-optimal version of the view, or some combination thereof. In some such embodiments, the output of the guidance machine learning model 610 includes a prediction of one or more manipulations to obtain a sub-optimal version of a view from an optimal version of the view. In such embodiments, the facility may invert the prediction of the one or more manipulations, such that the inverted manipulations yield an optimal version of the view from the sub-optimal version of the view. In some embodiments, the guidance machine learning model 610 is trained to invert the prediction of the one or more manipulations, such that the output of the guidance machine learning model 610 indicates one or more manipulations to yield an optimal version of the view from a sub-optimal version of the view. FIGS. 8A and 8B, described below, are examples of ultrasound images that may be used as training data for the guidance machine learning model 610.

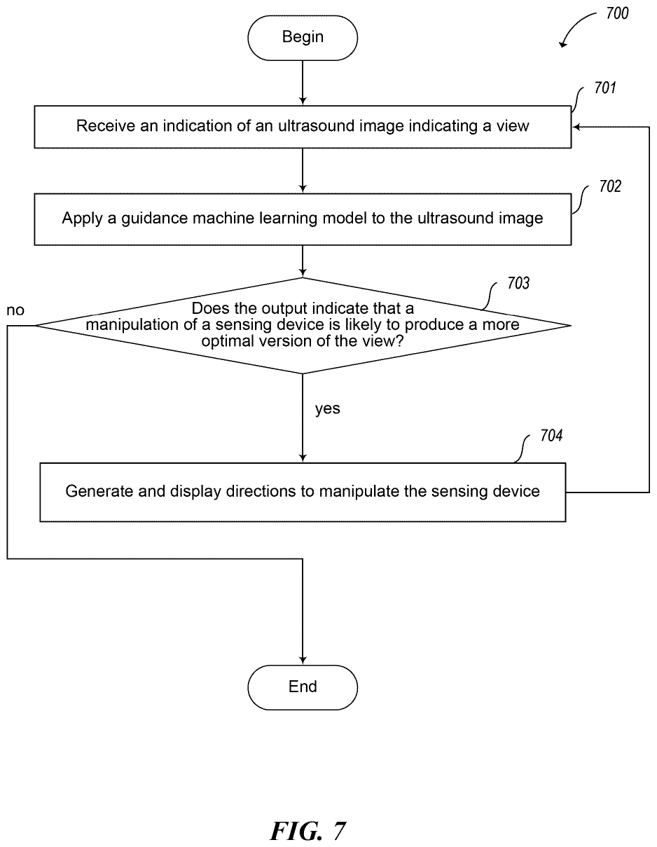

In some embodiments, the facility may apply a guidance machine learning model to the ultrasound image by using the process 700, described below in connection with FIG. 7.

FIG. 7 is a flow diagram showing a sample process 700 to use a guidance machine learning model to guide a clinician to produce an ultrasound image, used by the facility in some embodiments.

At act 701, the facility receives an indication of an ultrasound image that indicates a view associated with a diagnostic protocol.

At act 702, the facility applies a guidance machine learning model to the ultrasound image.

At act 703, the facility determines whether the output of the guidance machine learning model indicates that a manipulation of a sensing device of the ultrasound system is likely to produce a more optimal version of the view. If the output of the guidance machine learning model does not indicate that a manipulation of the sensing device is likely to produce a more optimal version of the view, the process 700 ends, otherwise, the process 700 proceeds to act 704.

At act 704, the facility generates and displays directions to manipulate the sensing device. In some embodiments, the facility generates the directions to manipulate the sensing device based on the output of the guidance machine learning model. After act 704, the process 700 proceeds to act 701.

While FIG. 8A and each of the ultrasound image diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

FIG. 8A is a sample ultrasound image 800 for training a guidance machine learning model, used by the facility in some embodiments. The ultrasound image 800 indicates an optimal version of a view of the Apical 4 Chamber of a patient's heart.

FIG. 8B is a sample ultrasound image 850 for training a guidance machine learning model, used by the facility in some embodiments. The ultrasound image 850 indicates a sub-optimal version of a view of the Apical 4 Chamber of a patient's heart where the head of a probe of an ultrasound transducer is pointed upwards relative to the optimal position of the probe.

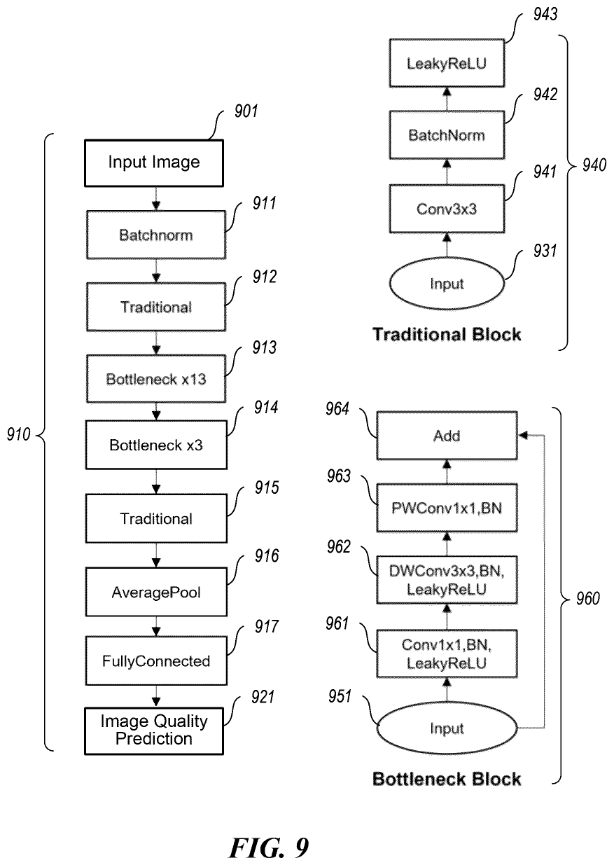

Returning to FIG. 3, at act 306, the facility determines whether at least one produced ultrasound image indicates an optimal version of the selected view. If the facility determines that at least one produced ultrasound image indicates an optimal version of the selected view, the facility automatically captures and stores the at least one produced ultrasound image and the process 300 proceeds to act 307, otherwise the process 300 proceeds to act 305. In some embodiments, the facility performs act 306 by applying a grading machine learning model, such as the grading machine learning model 910 described below in connection with FIG. 9, to at least one produced ultrasound image.

FIG. 9 is a model architecture diagram showing the organization of a classification machine learning model for grading an ultrasound image 910 (a “grading machine learning model”), used by the facility in some embodiments. The grading machine learning model 910 includes a batch normalization block 911, one or more traditional blocks 912 and 915, one or more bottleneck blocks 913 and 914, an average pooling layer 916, and a fully connected layer 917. The traditional blocks 912 and 915 may each comprise a traditional block 940. The traditional block 940 includes an input 911, a convolutional block 941, a batch normalization block 942, and a leaky ReLU activation function block 943. The bottleneck blocks 913 and 914 may each comprise a bottleneck block 960. The bottleneck block 960 includes an input 951, a convolutional bottleneck and leaky ReLU activation function layer 961, a depthwise convolutional bottleneck and leaky ReLU activation function layer 962, a pointwise convolutional bottleneck layer 963, and one or more additional layers 964. In some embodiments, the grading machine learning model 910 includes a convolutional neural network feature extractor (not shown) that extracts one or more features of ultrasound images received by the grading machine learning model 910 as input.

The grading machine learning model 910 may receive an ultrasound image 901 as input. In some embodiments, the grading machine learning model 910 receives one or more other types of ultrasound echo data as input, such as images, videos, sequences of images, other ultrasound artifacts or ultrasound echo data, or some combination thereof.

The grading machine learning model 910 outputs a prediction 921. In some embodiments, the prediction 921 may include a prediction of a score that indicates whether the ultrasound image depicts an optimal version of a view, a sub-optimal version of a view, or an version of the view that is not usable for the diagnostic protocol. In some embodiments, the facility determines whether the score output by the grading machine learning model 916 is within a threshold range of a score for an ultrasound image that represents an optimal version of the view. In some such embodiments, the facility determines whether the ultrasound image depicts a version of the view that is to be captured for diagnostic purposes based on the determination of whether the score is within the threshold range of the score for an ultrasound image that represents an optimal version of the view. In such embodiments, the facility captures the ultrasound image as an ultrasound image that represents a version of the view that can be used to diagnose a patient based on the determination of whether the score is within the threshold range, such as by storing the ultrasound image. In situations where the score is not within the threshold range, the facility may apply the ultrasound image to a guidance machine learning model, such as the guidance machine learning model 610 described above in connection with FIG. 6, to generate one or more directions to a clinician to manipulate one or more aspects of the ultrasound system to produce a view that can be used to diagnose a patient.

In some embodiments, the grading machine learning model 910 is trained by using a set of ultrasound images and a set of scores for each ultrasound image. In such embodiments, at least one ultrasound image and one score corresponding to the ultrasound image used to train the grading machine learning model 910 indicate an optimal version of a view and a score of the optimal version of the view.

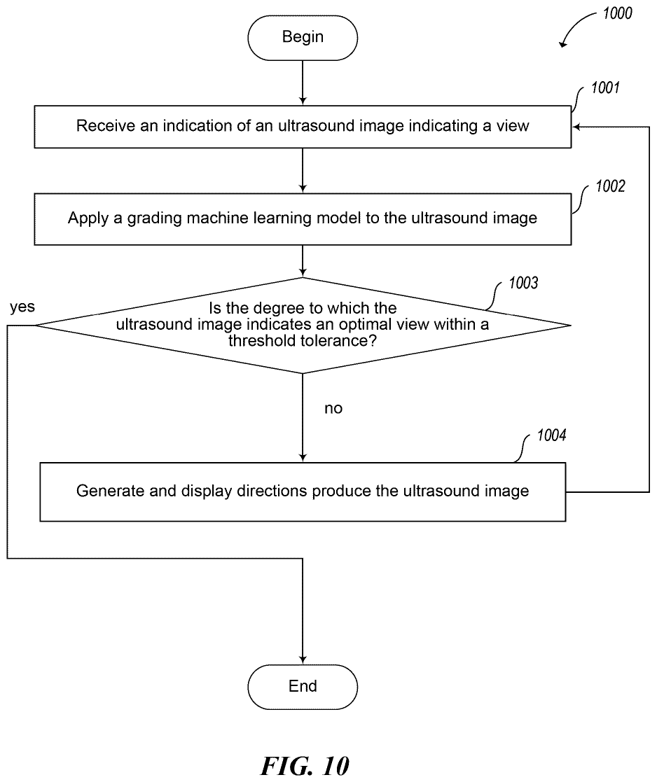

In some embodiments, the facility may apply a grading machine learning model to at least one produced ultrasound image by using the process 1000, described below in connection with FIG. 10.

FIG. 10 is a flow diagram showing a sample process 1000 to use a grading machine learning model for automatically capturing ultrasound images, used by the facility in some embodiments.

First, at act 1001, the facility receives an indication of an ultrasound image that indicates a view associated with a diagnostic protocol.

At act 1002, the facility applies a grading machine learning model to the ultrasound image.

At act 1003, the facility determines whether the degree to which the ultrasound image indicates an optimal view is within a threshold tolerance based on the output of the grading machine learning model. If the output of the grading machine learning model indicates that the degree to which the ultrasound image indicates an optimal view is within the threshold tolerance, the process 1000 ends, otherwise the process 1000 proceeds to act 1004.

At act 1004, the facility generates and displays directions to produce the ultrasound image. In some embodiments, the facility generates directions to produce the ultrasound image by applying a guidance machine learning model to the ultrasound image. After act 1004, the process 1000 proceeds to act 1001.

Returning to FIG. 3, at act 307, the facility determines whether one or more ultrasound images for at least one view associated with the diagnostic protocol have not yet been captured. If the facility determines that one or more ultrasound images for at least one view associated with the diagnostic protocol have not yet been captured, the process 300 proceeds to act 302, otherwise the process 300 ends.

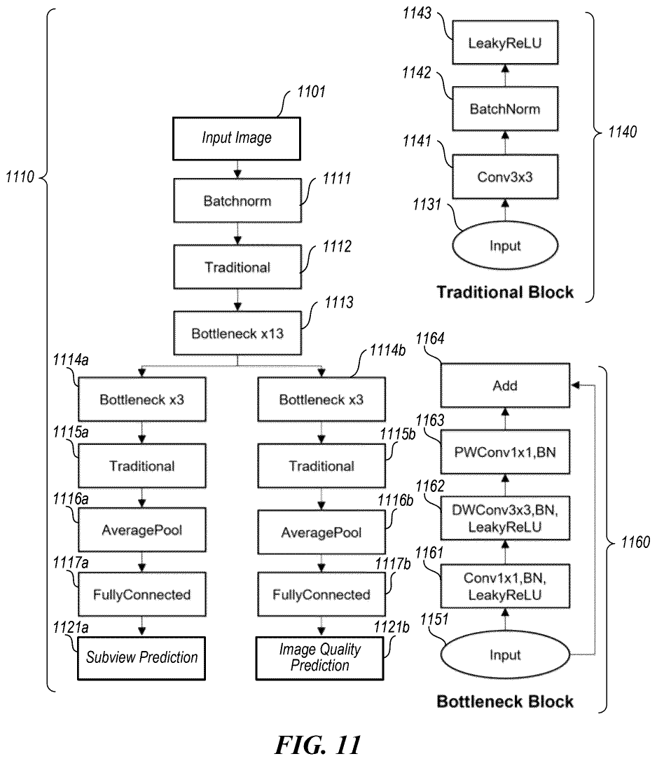

In some embodiments, the facility may select one or more machine learning models associated with a selected view, anatomical window, diagnostic protocol, or some combination thereof. In such embodiments, the facility may use the selected machine learning models to perform one or more of the acts described above in connection with the processes 300, 500, 800, and 1000. In some embodiments, one or more machine learning models used to perform one or more of the acts described above in connection with the process 300, 500, 800, and 1000, are able to perform the functions of two or more of the guidance machine learning model, grading machine learning model, and anatomical window machine learning model. For example, in some embodiments, the facility may use a grading and guidance machine learning model that determines whether an ultrasound image indicates a selected view, whether one or more manipulations are likely to produce an optimal version of the selected view, and whether the ultrasound image is sufficient for diagnosing a patient, such as the combination grading and guidance classification machine learning model 1110 described below in connection with FIG. 11.

FIG. 11 is a model architecture diagram showing the organization of a combination grading and guidance classification machine learning model 1110, used by the facility in some embodiments. The grading and guidance classification machine learning model 1110 includes a batch normalization block 1111, one or more traditional blocks 1112, 1115a, and 1115b, one or more bottleneck blocks 1113, 1114a, and 1114b, one or more average pooling layers 1116a and 1116b, and one or more fully connected layers 1117a and 1117b. The traditional blocks 1112, 1115a, and 1115b may each comprise a traditional block 1140. The traditional block 1140 may be similar to the traditional blocks 640 and 940, described above in connection with FIGS. 6 and 9 respectively. The bottleneck blocks 1113, 1114a, and 1114b may each comprise a bottleneck block 1160. The bottleneck block 1160 may be similar to the bottleneck blocks 660 and 960, described above in connection with FIGS. 6 and 9 respectively. In some embodiments, the grading and guidance machine learning model 1110 includes a convolutional neural network feature extractor (not shown) that extracts one or more features of ultrasound images received by the grading and guidance machine learning model 1110 as input.

The grading and guidance classification machine learning model 1110 may receive input 1101 in a similar manner to the guidance machine learning model 610 and grading machine learning model 910, described above in connection with FIGS. 6 and 9 respectively. The grading and guidance classification machine learning model 1110 may output a prediction 1121a similar to the prediction 621 of the guidance machine learning model 610, described above in connection with FIG. 6. The grading and guidance classification machine learning model 1110 may also output a prediction 1121b that is similar to the prediction 921 of the grading machine learning model 910, described above in connection with FIG. 9. The grading and guidance classification machine learning model 1110 may be trained with training data similar to the training data used to train the guidance machine learning model 610 and grading machine learning model 910, described above in connection with FIGS. 6 and 9 respectively.

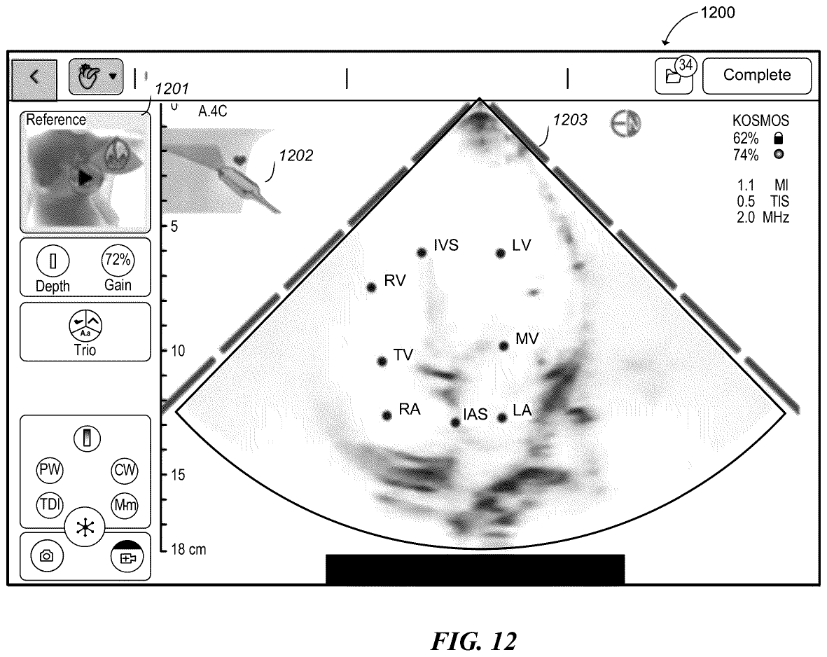

FIG. 12 is a display diagram showing a sample ultrasound image 1200 output by the facility for directing a clinician in some embodiments. The sample ultrasound image 1200 includes an anatomical window user interface element 1201, a guidance user interface element 1202, and a grading user interface element 1203. The anatomical window user interface element 1201 indicates one or more anatomical windows associated with a diagnostic protocol. In some embodiments, the facility may use the anatomical window user interface element 1201 to display one or more directions for manipulating an aspect of the ultrasound system to produce ultrasound images depicting one or more views for an anatomical window associated with a diagnostic. The guidance user interface element 1202 indicates one or more manipulations for capturing an optimal version of a view associated with a diagnostic protocol. In some embodiments, the facility may use the guidance user interface element 1202 to display one or more directions for manipulating an aspect of the ultrasound system to produce an optimal version of a view associated with a diagnostic protocol, such as one or more directions received as output from a guidance machine learning model. In some embodiments, the facility displays the directions in the form of one or more images, animated images, videos, text, or other forms of displaying directions. In this example, because the ultrasound image includes an optimal version of the selected view, the guidance user interface element 1202 indicates that no probe manipulation is required.

The grading user interface element 1203 indicates a score that represents the level to which an ultrasound image produced by the ultrasound system is an optimal version of a view for a diagnostic protocol, such as a score received as output from a grading machine learning model. For example, the grading user interface element 1203 displays five line segments on the left and right side of the ultrasound image depicted in FIG. 12, which indicates that the ultrasound image received a score of five. In this example, such a score indicates that the ultrasound image includes an optimal version of a selected view. In some embodiments, the grading user interface element 1203 includes one or more visual representations of a score, such as a color, a number of lines or other shapes, a number, other visual representations of a score, or some combination thereof. In an example embodiment, the grading user interface element 1203 changes color relative to a score assigned to an ultrasound image. In another example embodiment, the grading user interface element 1203 displays one or more shapes around a displayed ultrasound image that represent a score of the ultrasound image.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system comprising:
an ultrasound sensing device; and
a computing device, the computing device comprising:
a communication interface configured to directly receive ultrasound echo data sensed by the ultrasound sensing device from a patient, the received ultrasound echo data comprising a sequence of ultrasound images;
a memory configured to:
store one or more machine learning models trained to analyze the sequence of ultrasound images; and
store one or more representations of one or more diagnostic protocols, each representation of a diagnostic protocol indicating one or more views;
a processor configured to:
apply the one or more machine learning models to one or more first ultrasound images of the sequence of ultrasound images;
identify a first view of the diagnostic protocol based on the output of the one or more machine learning models; and
generating one or more directions to capture an ultrasound image associated with the first view based on the output of the one or more machine learning models;
a display device configured to:
display an indication of the generated one or more directions to capture the ultrasound image associated with the first view.

2. The system of claim 1, the one or more machine learning models comprise at least one of:
a machine learning model configured to output a score representing the degree to which an ultrasound image indicates an optimal view for a diagnostic protocol;
a machine learning model configured to output a manipulation of a sensing device likely to produce a more optimal version of a view to which an ultrasound image corresponds; and
a machine learning model configured to output whether an ultrasound image was captured near an anatomical window.

3. The system of claim 1, wherein at least one machine learning model of the one or more machine learning models outputs whether an ultrasound image was captured near at least one anatomical window associated with a diagnostic protocol,
the processor being further configured to:
apply the at least one machine learning model to ultrasound echo data associated with the first view; and
determine whether the ultrasound echo data constitutes an ultrasound image captured near an anatomical window including a region associated with the first view based on the output of the machine learning model.

4. The system of claim 1, wherein, at least one machine learning model of the one or more machine learning models outputs a manipulation of a sensing device likely to produce a more optimal version of a view to which an ultrasound image corresponds, the processor being further configured to:
apply the at least one machine learning model to the ultrasound echo data associated with the first view; and
determine whether a manipulation of a sensing device is likely to produce a more optimal version of the first view based on the output of the machine learning model.

5. The system of claim 4, wherein, based on a determination a manipulation of a sensing device is likely to produce a more optimal version of the first view based, the processor being further configured to:
identify one or more manipulations of a sensing device for capturing additional ultrasound echo data associated with the first view of the diagnostic protocol; and
cause the display device to display an indication of the one or more motions.

6. The system of claim 1, wherein at least one machine learning model of the one or more machine learning models outputs a score representing the degree to which an ultrasound image indicates an optimal view for a diagnostic protocol,
the processor being further configured to:
apply the at least one machine learning model to the ultrasound echo data associated with the first view to receive a score representing the degree to which an ultrasound image indicates an optimal view for the first view of the diagnostic protocol; and
determining whether to store the ultrasound image based on the score output by the at least one machine learning model.

7. The system of claim 1, wherein the processor is further configured to:
select at least one machine learning model of the one or more machine learning models based on a second view and the diagnostic protocol; and
apply the selected at least one machine learning model to the sequence of ultrasound images.

8. The system of claim 1, wherein the processor is further configured to:
receive input selecting the diagnostic protocol;
select at least one machine learning model of the one or more machine learning models based on the selected the diagnostic protocol; and
apply the selected at least one machine learning model to the sequence of ultrasound images.

9. One or more instances of computer-readable media collectively having contents configured to cause a computing system to perform a diagnostic method with respect to a person, the method comprising:
accessing a set of machine learning models;
receiving a sequence of ultrasound images;
applying at least one machine learning model to one or more first ultrasound images of the sequence of ultrasound images;
identifying a first view of the diagnostic protocol based on the output of the one or more machine learning models; and
generating one or more directions to capture an ultrasound image associated with the first view based on the output of the one or more machine learning models.

10. The one or more instances of computer-readable media of claim 9, wherein the at least one machine learning model of the set of machine learning models outputs whether an ultrasound image was captured near at least one anatomical window associated with the diagnostic protocol, the method further comprising:
applying the at least one machine learning model to the sequence of ultrasound images; and
determining whether an ultrasound image was captured near an anatomical window including a region associated with the first view based on the output of the machine learning model.

11. The one or more instances of computer-readable media of claim 9, wherein the at least one machine learning model of the set of machine learning models outputs a manipulation of a sensing device likely to produce a more optimal version of a view to which an ultrasound image corresponds, method further comprising:
applying the at least one machine learning model to the sequence of ultrasound images; and
determining whether a manipulation of a sensing device is likely to produce a more optimal version of the first view based on the output of the machine learning model.

12. The one or more instances of computer-readable media of claim 11, wherein, based on a determination a manipulation of a sensing device is likely to produce a more optimal version of the first view based, the method further comprises:
identifying one or more manipulations of a sensing device for capturing ultrasound echo data associated with the first view of the diagnostic protocol; and
causing the display device to display an indication of the one or more motions.

13. The one or more instances of computer-readable media of claim 9, wherein the at least one machine learning model of the set of machine learning models outputs a score representing the degree to which an ultrasound image indicates an optimal view for a diagnostic protocol, the method further comprising:
applying the at least one machine learning model to the first ultrasound images to receive a score representing the degree to which an ultrasound image included in the first ultrasound images indicates an optimal view for the first view of the diagnostic protocol; and
determining whether to store the ultrasound image based on the score output by the at least one machine learning model.

14. The one or more instances of computer-readable media of claim 9, the method further comprising:
selecting one or more machine learning models of the set of machine learning models based on a second view and the diagnostic protocol; and
applying the selected one or more machine learning models to the sequence of ultrasound images.

15. The one or more instances of computer-readable media of claim 9, the method further comprising:
receiving input selecting the diagnostic protocol;
selecting one or more machine learning models of the set of machine learning models based on the selected the diagnostic protocol; and
applying the selected one or more machine learning models to the sequence of ultrasound images.

16. A diagnostic method performed by a computing system with respect to a person, the diagnostic method comprising:
accessing a set of machine learning models;
receiving a sequence of ultrasound images;
applying at least one machine learning model to one or more first ultrasound images of the sequence of ultrasound images;
identifying a first view of the diagnostic protocol based on the output of the one or more machine learning models; and
generating one or more directions to capture an ultrasound image associated with the first view based on the output of the one or more machine learning models.

17. The diagnostic method of claim 16, wherein the at least one machine learning model of the set of machine learning models outputs whether an ultrasound image was captured near at least one anatomical window associated with the diagnostic protocol, the method further comprising:
applying the at least one machine learning model to the sequence of ultrasound images; and
determining whether an ultrasound image was captured near an anatomical window including a region associated with the first view based on the output of the machine learning model.

18. The diagnostic method of claim 16, wherein the at least one machine learning model of the set of machine learning models outputs a manipulation of a sensing device likely to produce a more optimal version of a view to which an ultrasound image corresponds, method further comprising:
applying the at least one machine learning model to the sequence of ultrasound images; and
determining whether a manipulation of a sensing device is likely to produce a more optimal version of the first view based on the output of the machine learning model.

19. The diagnostic method of claim 18, wherein, based on a determination a manipulation of a sensing device is likely to produce a more optimal version of the first view based, the method further comprises:
identifying one or more manipulations of a sensing device for capturing ultrasound echo data associated with the first view of the diagnostic protocol; and
causing the display device to display an indication of the one or more motions.

20. The diagnostic method of claim 16, wherein the at least one machine learning model of the set of machine learning models outputs a score representing the degree to which an ultrasound image indicates an optimal view for a diagnostic protocol, the method further comprising:
applying the at least one machine learning model to the first ultrasound images to receive a score representing the degree to which an ultrasound image included in the first ultrasound images indicates an optimal view for the first view of the diagnostic protocol; and
determining whether to store the ultrasound image based on the score output by the at least one machine learning model.

21. The diagnostic method of claim 16, the method further comprising:
selecting one or more machine learning models of the set of machine learning models based on a second view and the diagnostic protocol; and
applying the selected one or more machine learning models to the sequence of ultrasound images.

22. The diagnostic method of claim 16, the method further comprising:
receiving input selecting the diagnostic protocol;
selecting one or more machine learning models of the set of machine learning models based on the selected the diagnostic protocol; and
applying the selected one or more machine learning models to the sequence of ultrasound images.
